(19) United States
(12) Patent Application Publication
(10) Pub. No.: US 2025/0221806 A1
(43) Pub. Date: Jul. 10, 2025

(54) SOFT TISSUE IMPLANT

(71) Applicant: Cornell University, Ithaca, NY (US)

(72) Inventors: Jason Spector, Ithaca, NY (US); Luke Poveromo, Ithaca, IN (US)

(21) Appl. No.: 18/854,440

(22) PCT Filed: Apr. 5, 2023
(86) PCT No.: PCT/US23/17515

§ 371 Date: Oct. 4, 2024

Related U.S. Application Data
(60) Provisional application No. 63/327,742, filed on Apr. 5, 2022.

(51) Int. Cl. A61F 2/00; A61F 2/12

(52) U.S. Cl. CPC A61F 2/0059; A61F 2/0077; A61F 2/12; A61F 2002/0086

(57) ABSTRACT

An implant including a shell and a plurality of position-stabilizing wells is described. The position-stabilizing wells are defined by an opening at least one-half (0.5) millimeters across. The position-stabilizing wells allow the ingrowth of tissue so as to stabilize the shaped in implant after implantation in a patient. Additionally, the shaped implant has a smooth surface so as to discourage the formation of a bacterial biofilm. The disclosed implant can further be attached to an underlying tissue in the patient by position-stabilizing tabs.

### PRIORITY CLAIM

This application claims priority to U.S. Provisional Application Ser. No. 63/372,742, filed 5 Apr. 2023, which is expressly incorporated by reference herein.

### BACKGROUND

Textured implants have been typically used for breast reconstruction because unlike smooth implants, a rough surface of the textured implant provides positional stability by resisting rotation of the implant after it is placed in a patient. This allows for the implants to be shaped to closely mimic the breast tissue. Additionally, textured implants have been shown to cause decreased rates of capsular contracture, implant malposition, and reoperation, and improve overall aesthetic outcomes. However, the textured implants have been increasingly associated with Breast Implant Associated-Acute Large Cell Lymphoma (BIA-ALCL) and have recently been recommended for voluntary recall by the FDA. The leading theory associated with the underlying etiology of BIA-ALCL is that the rough surface of the textured implants provides a shelter for a bacterial biofilm triggering a chronic inflammatory response that ultimately becomes malignant causing BIA-ALCL.

Accordingly, there is a need to develop implants that can provide stability upon implantation without harboring bacterial biofilms than may trigger an inflammatory response in the patient.

### SUMMARY

The presently disclosed implant that includes a shell and a plurality of position-stabilizing wells provides implant stability and discourages bacterial biofilm formation after implantation in a patient. This specific design of the implant negates the need for a textured implant surface topography, which has been causatively associated with BIA-ALCL. Thus, the implant disclosed is advantageous over currently available smooth and/or textured implants.

The implants include position-stabilizing wells and are shaped to anatomically resemble breast tissue. The presence of the position-stabilizing wells allows tissue to grow into the implant and stabilize the implant in its implantation position. The position-stabilizing wells are defined by an opening at least 0.5 millimeter across. The shape, size, and number of the position-stabilizing wells in the implant can be varied to provide the required stability to the implant.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a schematic of a breast implant positioned in a patient;

FIG. 2 is an exploded view of the breast implant of FIG. 1 showing an asymmetrical shell and a plurality of position-stabilizing wells;

FIG. 3 is a side view of the breast implant showing the shell having an exterior surface, a distal panel, a proximal panel, filler, and a plurality of position-stabilizing wells;

FIG. 4 is a scanning electron micrograph image of the exterior surface of the shell of the breast implant of FIG. 3;

FIG. 5 is an exploded view of a cylindrically shaped position-stabilizing well defined by an opening, side surface, and bottom surface;

FIG. 6 is an exploded view of a conically shaped position-stabilizing well defined by an opening and side surface;

FIG. 7 is an exploded view of a polygonal shaped position-stabilizing well denied by an opening, side surface, and bottom surface;

FIG. 8 is an exploded view of a frustum shaped position-stabilizing well defined by an opening, side surface, and bottom surface;

FIG. 9. is an embodiment of an implant with an optional position-stabilizing tab;

FIG. 10 is a method of implanting the implant of FIG. 3 in the patient;

FIG. 11 is an illustration of spherical implants with position-stabilizing wells that are 2 mm in width and 2 mm in height, implants with position-stabilizing wells that are 4 mm in width and 2 mm in height, and implants with position-stabilizing wells that are 2 mm in width and 4 mm in height in a rat model;

FIG. 12A is a schematic showing the location of the spherical implants of FIG. 11 in the rat model at implantation;

FIG. 12B is a schematic showing the location of the spherical implants of FIG. 11 in the rat model at one month post-implantation;

FIG. 13 is a graph showing the mean positional rotation of the implants of FIG. 11 one month after they were implanted in the rat model;

FIG. 14 is an illustration of H&E staining of capsules surrounding the implants of FIG. 11 after one month.

FIG. 15 is a graph showing capsule thickness one month after the implants of FIG. 11 were implanted in the rat model;

FIG. 16 is an illustration of spherical implants with 22 position-stabilizing wells that are 2 mm in width and 2 mm in height, implants with 22 position-stabilizing wells that are 1 mm in width and 2 mm in height, implants with 22 position-stabilizing wells that are 2 mm in width and 3 mm in height, implants with 11 position-stabilizing wells that are 4 mm in width and 2 mm in height, and implants with 11 position-stabilizing wells that are 2 mm in width and 2 mm in height.

FIG. 17A is a schematic showing the location of the spherical implants of FIG. 16 in the rat model at implantation;

FIG. 17B is a schematic showing the location of the spherical implants of FIG. 16 in the rat model at one month post-implantation;

FIG. 17C is a schematic showing the location of the spherical implants of FIG. 16 in the rat model at three months post-implantation;

FIG. 18 is a graph showing mean positional rotation of the implants of FIG. 16 three months after they were implanted in the rat model;

FIG. 19 is a graph showing the mean positional rotation of the implants of FIG. 16 as a function of time for a period of 3 months after they were implanted in the rat model;

FIG. 20 is an illustration of H&E staining of capsules surrounding the implants of FIG. 16 after three months;

FIG. 21 is a graph showing capsule thickness one month after the implants of FIG. 16 were implanted in the rat model;

FIG. 22 is an illustration of spherical implants with position-stabilizing tabs and position-stabilizing wells; specifically, implants with 22 position-stabilizing wells that are 2 mm in width and 2 mm in height, implants with 22 position-stabilizing wells that are 1 mm in width and 2 mm in height, implants with 22 position-stabilizing wells that are 2 mm in width and 3 mm in height, implants with 11 position-stabilizing wells that are 4 mm in width and 2 mm in height, and implants with 11 position-stabilizing wells that are 2 mm in width and 2 mm in height are shown;

FIG. 23A is a graph showing mean positional rotation of the implants of FIG. 22 two weeks after they were implanted in the rat model;

FIG. 23B is a graph showing mean positional rotation of the implants of FIG. 22 four weeks after they were implanted in the rat model;

FIG. 24 is an illustration of a smooth implant without any position-stabilizing wells;

FIG. 25 is a scanning electron micrograph image of the exterior surface of the shell of the implant of FIG. 24;

FIG. 26 is an illustration of a textured implant without any position-stabilizing wells; and

FIG. 27 is a scanning electron micrograph image of the exterior surface of the shell of the implant of FIG. 26.

### DETAILED DESCRIPTION

The present disclosure relates to embodiments of an implant that can be used for tissue reconstruction, including but not limited to breast reconstruction. Specifically, the present disclosure relates to embodiments of an implant that can restore the benefits of textured implants while circumventing the risk of Breast Implant Associated-Acute Large Cell Lymphoma (BIA-ALCL) in a patient.

As discussed in detail herein, an asymmetrical breast implant 10 resembling native breast tissue can be stably implanted in a patient while discouraging bacterial biofilm formation, which can cause BIA-ALCL, infection, and/or capsular contracture. The implants 10 include a plurality of position-stabilizing wells 16 defined by an opening at least 0.5 millimeter across that allow an ingrowth of tissue. Such ingrowth of tissue into the position-stabilizing wells 16 stabilizes the implant 10 after implantation in the patient. Furthermore, the implant 10 and the position-stabilizing wells 16 include a smooth exterior surface, which discourages the formation of bacterial biofilm hypothesized to be causatively linked to BIA-ALCL.

Certain exemplary embodiments will now be described to provide an overall understanding of the principles of the disclosure. One or more examples of these embodiments are illustrated in the accompanying drawings.

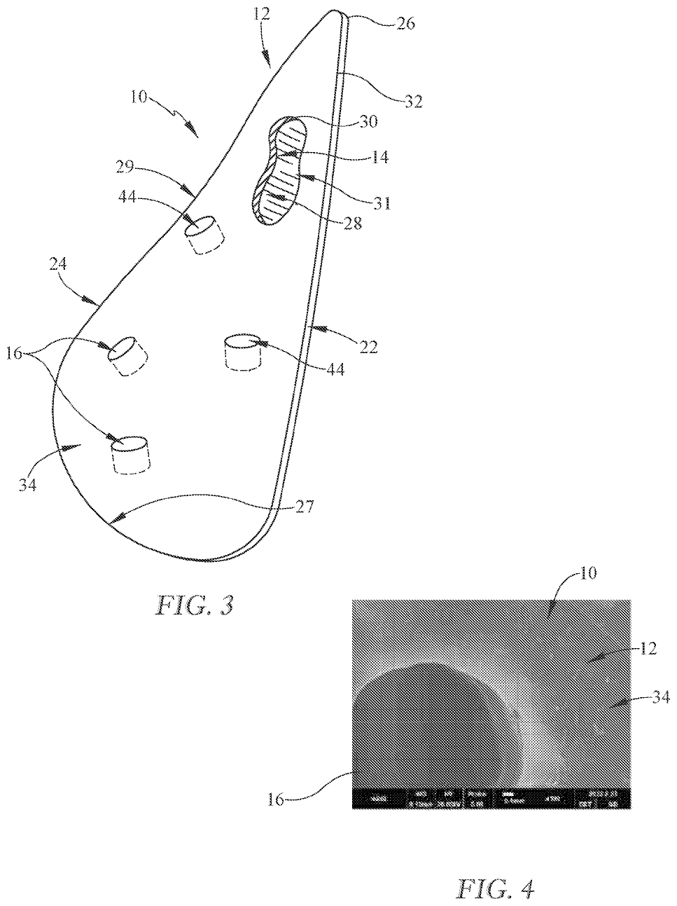

An exemplary soft tissue implant is illustrated as a breast implant 10 in FIG. 3. The implant 10 is a shaped implant and includes a shell 12, a gel or other filler 14, and a plurality of position-stabilizing wells 16.

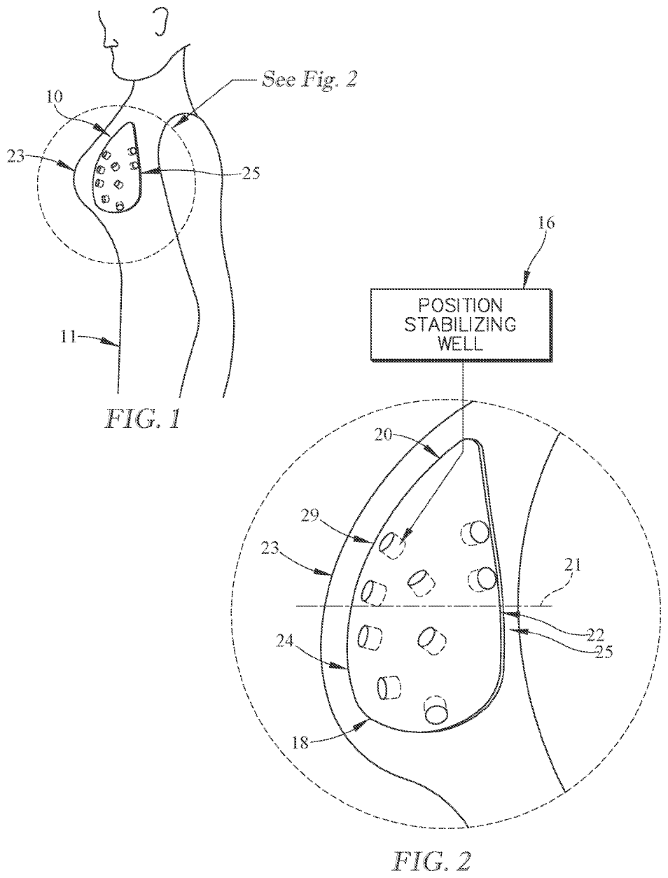

The shell 12 includes a bottom portion 18 that is wider than a top portion 20 so that when the implant 10 is placed in a patient 11 as shown in FIGS. 1 and 3, the top portion 20 remains substantially above the bottom portion 18. The shell 12 is asymmetrical from the bottom portion 18 to the top portion 20 relative to a horizontal axis 21 across the midsection of the shell 12 as shown in FIG. 2.

In other embodiments, the shell 12 may be round symmetrical shape when incorporated into a round implant. In such embodiments, the position stabilizing wells 16 fix the implant relative to surrounding tissue.

The illustrative shell 12 includes a proximal panel 22, a distal panel 24, and a fusion interface 26 as shown in FIG. 3. The distal panel 24 is configured to be anatomically shaped so as to represent human breast tissue. The proximal panel 22 is configured to be a flat sheet to maximize contact with an underlying tissue 25 as shown in FIG. 2. The shell 12 is fabricated by fusing a concave side 27 of the proximal panel 22 with the distal panel 24 at the fusion interface 26 to define an interior space 28 as shown in FIG. 3. A convex side 29 of the distal panel 24 is configured to contact the skin 23 of the patient 11 while the proximal panel 22 is configured to contact the muscle 25 of the patient 11 upon implantation of the implant 10 as shown in FIGS. 1 and 2. Of course, other known implementations of breast implants are also contemplated in which the implant 10 may be arranged or sandwiched between a chest wall and a pectoral muscle; a pectoral muscle and natural breast tissue; and/or a pectoral muscle and skin. In some embodiments, portions of the implant 10 can be between different anatomical features such as with an upper portion between a breast plate and a pectoral muscle, while a lower portion is between a pectoral muscle and natural breast tissue or skin.

As shown in a cut-out region 31, the gel 14 is comprised in the interior space 28 defined by the shell 12 as shown in FIG. 3. The gel 14 is configured to provide compliance to the implant 10. The gel 14 may be provided by a silicone based material and/or a saline based material. Further, in applications in which the implant is used as a temporary tissue expander, the gel 14 may be replaced by air. Of course, other suitable fillers may also be used as (or in place of) the gel 14.

In the illustrative example, FIG. 3, the distal panel 24 of the shell has a thickness 30 that is larger than a thickness 32 of the proximal panel 22. In some embodiments, the distal panel 24 of the shell may have a thickness 30 that is smaller than the thickness 32 of the proximal panel 26. In other embodiments, the distal panel 24 of the shell may have a thickness 30 that is equal to the thickness 32 of the proximal panel 22.

Typically available breast implants as shown in FIG. 26 are textured implants 313. The irregular surface area of the textured implant 313, as illustrated in the electron scanning microscopy image (see FIG. 27) of the textured implant 313, promotes persistence of a bacterial biofilm which continually interacts with the immune system of the patient 11 and triggers Breast Implant Associated-Acute Large Cell Lymphoma (BIA-ALCL). For example, bacterial biofilm is known to form in implants with textured surfaces that have a roughness of greater than about 4 μm (Jacombs et al. (2014); James et. al. (2019); Collett et. al., (2019)). Thus, bacterial biofilm formation is managed by configuring the implant 10 to have a shell 12 with a smooth outermost surface 34. As shown by the scanning electron microscopy image in FIG. 4, the outermost surface 34 of the shell 12 is smooth and has a surface roughness of less than or equal to about 4 μm so as to discourage development of bacterial biofilm. In some embodiments, the outermost surface 34 of the shell 12 has a surface roughness of less than or equal to about 3 μm. In other embodiments, the outermost surface 34 of the shell 12 has a surface roughness of less than or equal to about 2 μm.

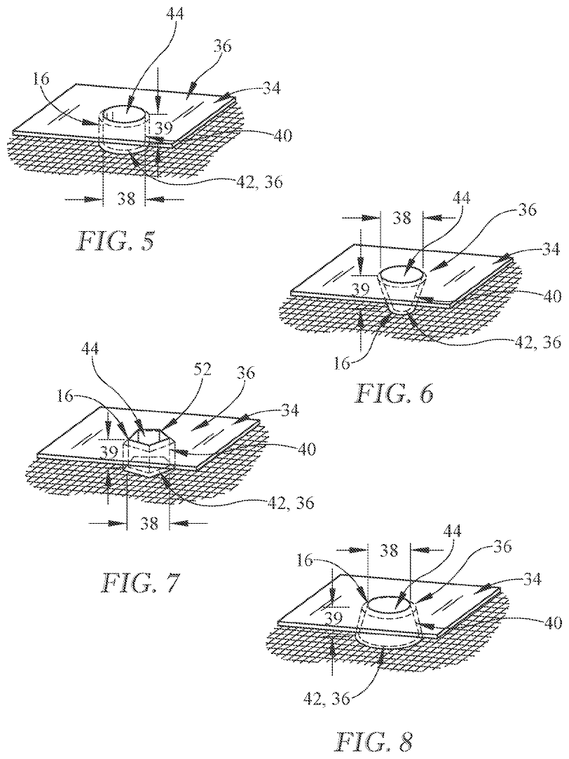

As shown in FIG. 2, the implant 10 includes a plurality of position-stabilizing wells 16 on the proximal panel 22 and on the distal panel 24. Each position-stabilizing well 16 extends from the outermost surface 34 of the shell 12 inward towards the gel 14. The position-stabilizing well 16 includes an opening 38, a side surface 40, and a bottom surface 42, as shown in FIGS. 5-8. An exterior surface 36 of the shell 12 includes the outermost surface 34 of the shell 12, the side surface 40 of each position-stabilizing well 16, and the bottom surface 42 of each position-stabilizing well 16. The shell 12 includes an innermost surface 43 that comprises the bottom surface 42 of the position-stabilizing wells 16.

The opening 38 of each positional stabilizing-well 16 may range from about 0.5 mm to about 10 mm, including any size or range comprised therein. For example, the opening 38 may be about 0.5 mm to about 2 mm, about 2 mm to about 4 mm, about 4 mm to about 6 mm, about 6 mm to about 10 mm. In some embodiments, the opening 38 may be bigger than about 10 mm or smaller than about 0.5 mm.

Each position-stabilizing well 16 is pressed into the proximal panel 22 or into the distal panel 24 and is defined by a height 39. Each position-stabilizing well 16 has a uniform thickness equal to the thickness 32 of the proximal panel 22 or thickness 30 of the distal panel 24 along all surfaces 40, 42 of the position stabilizing well 16. The concave side 27 of the shell 12 underlying the position-stabilizing well 16 protrudes into the interior space 28 of the implant, thereby projecting the shell 12 into the interior space 28 at the site of the position-stabilizing well 16. Such a structural change may provide structural integrity to the shell 12 by uniformly maintaining the thickness 30 and 32 of the shell 12.

In alternative embodiments, the height 39 of each position-stabilizing well 16 is less than the thickness of the panel 22, 24 where the position-stabilizing well 16 is located. For example, if the position-stabilizing well 16 is located on the proximal panel 22, its height is less than the thickness 32 of the proximal panel 22. Alternatively, if the position-stabilizing well 16 is located on the distal panel 22, its height is less than the thickness 30 of the distal panel 24.

The height 39 of each positional stabilizing-well 16 may range from about 0.5 mm to about 10 mm, including any size or range comprised therein. For example, the height 39 may be about 0.5 mm to about 2 mm, about 2 mm to about 4 mm, about 4 mm to about 6 mm, about 6 mm to about 10 mm. In some embodiments, the height 39 may be bigger than about 10 mm or smaller than about 0.5 mm.

An interior volume 44 of each position-stabilizing well is defined by its opening 38, side surface 40 and bottom surface 42. In the illustrative example shown in FIG. 3, each of the plurality of the position-stabilizing wells 16 in the implant 10 has the same interior volume 44. In alternative embodiments, the position-stabilizing wells 16 in the implant may have different interior volumes 44. For example, in some embodiments, the interior volume 44 of the position-stabilizing well 16 on the distal panel 24 of the shell 12 may be different from the interior volume 44 of the position-stabilizing well 16 on the proximal panel 22 of the shell 12.

As shown in FIGS. 4-8, the side surface 40 and the bottom surface 42 of each position-stabilizing well 16 are smooth and have a surface roughness of less than or equal to about 4 μm so as to discourage development of bacterial biofilm. In some embodiments, the side surface 40 and the bottom surface 42 have a surface roughness of less than or equal to about 3 μm. In other embodiments, the side surface 40 and the bottom surface 42 have a surface roughness of less than or equal to about 2 μm.

Each position-stabilizing well 16 is configured to stabilize the implant 10 by resisting rotation of the implant 10 relative to the patient 11 so that the bottom portion 18 of the shell 12 remains below the top portion 20 of the shell 12 substantially in a selected position relative to the patient 11. The implant may not rotate by more than about 20 degrees to about 100 degrees including any value or range of rotation comprised therein from an implantation position relative to the patient. For example, the implant may not rotate by more than about 20 degrees to about 40 degrees, about 40 degrees to about 60 degrees about 60 degrees to about 80 degrees, or about 80 degrees to about 100 degrees including any value or range of rotation comprised therein from the implantation position relative to the patient.

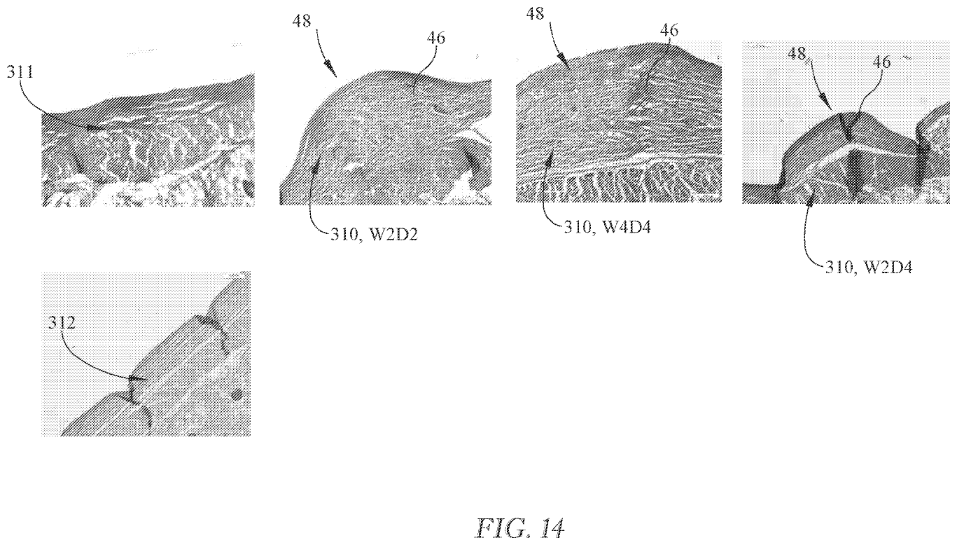

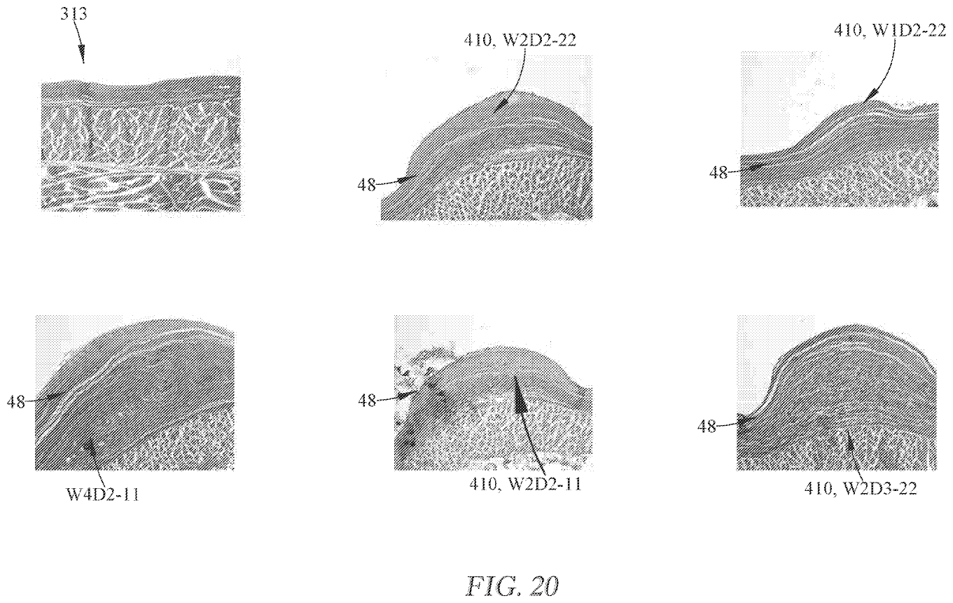

The position-stabilizing well 16 is configured to allow an ingrowth of tissue 46 when the implant 10 is placed in the patient 11. The ingrowth of tissue 46 in response to implantation results in the formation of a capsule 48 around the implant 10 as shown in FIGS. 14 and 20. An increase in the thickness of the capsule 48 in the interior volume 44 of the position-stabilizing well 16 results in an increase in the stabilization of the implant 10. The ingrowth of tissue 46 and the capsule 48 resist the rotation of the implant 10 relative to the patient 11.

The number of position-stabilizing wells 16 on the proximal panel 22 are equal to the number of position-stabilizing wells 16 on the distal panel 24. In other embodiments, the number of position-stabilizing wells 16 on the distal panel 24 may be more than the number of position-stabilizing wells 16 on the proximal panel 22. Since the distal panel 24 is curved to be representative of the anatomical shape of the breast tissue, the distal panel 24 has a larger surface area than the proximal panel 22. Thus, the implant 10 may include more position-stabilizing wells 16 on the distal panel 24 than on the proximal panel 22 so as to provide the required attachment density for the implant 10 to adhere to the skin 23.

In other embodiments, the number of position-stabilizing wells 16 on the proximal panel 22 may be more than the number on the distal panel 24. Since the proximal panel 22 is in contact with the muscle 25, more position-stabilizing wells 16 on the proximal panel 24 may be used to increase the strength of attachment of the implant 10 to the muscle 25 and stabilize the implant 10 relative to the patient 11.

The number of position-stabilizing wells 16 on the bottom portion 18 of the shell 12 are equal to the number on the top portion 20 of the shell. In other embodiments, the number of position-stabilizing wells 16 on the bottom portion 18 of the shell 12 may be more than the number on the top portion 20 of the shell 12. Since the bottom portion 18 is wider than the top portion 20, the bottom portion 18 has a larger surface area than the top portion 20. Thus, the implant 10 may include more position-stabilizing wells 16 on the bottom portion 18 so as to provide the required attachment density for the implant 10 to adhere to the skin 23.

The positional-stabilizing well 16 of the implant 10 may be of a cylindrical shape defined by the side surface 40 and the bottom surface 42 as shown in FIGS. 3 and 5. The interior volume 44 of the cylindrical shape is uniform and the ingrowth of the tissue 46 into the uniform interior volume 44 of the cylindrical positional-stabilizing well 16 provides a strong attachment of the implant 10 to the capsule 48.

The positional-stabilizing well 16 of the implant 10 may be of a conical shape defined by the side surface 40 as shown in FIG. 6. The interior volume 44 of the conical shape provides sufficient volume to allow ingrowth of the tissue 46 into the interior volume 44 while managing reduction in the shell thickness 30 and 32 and/or interior volume 44 closer to the gel 14.

The positional-stabilizing well 16 of the implant 10 may be of a polygonal shape defined by the side surface 40 and the bottom surface 42 as shown in FIG. 7. The polygonal shape provides more than one face 52 that resists rotation of the tissue 46 within the position-stabilizing well 16 and minimizes removal of the tissue 46 from the position-stabilizing well 16.

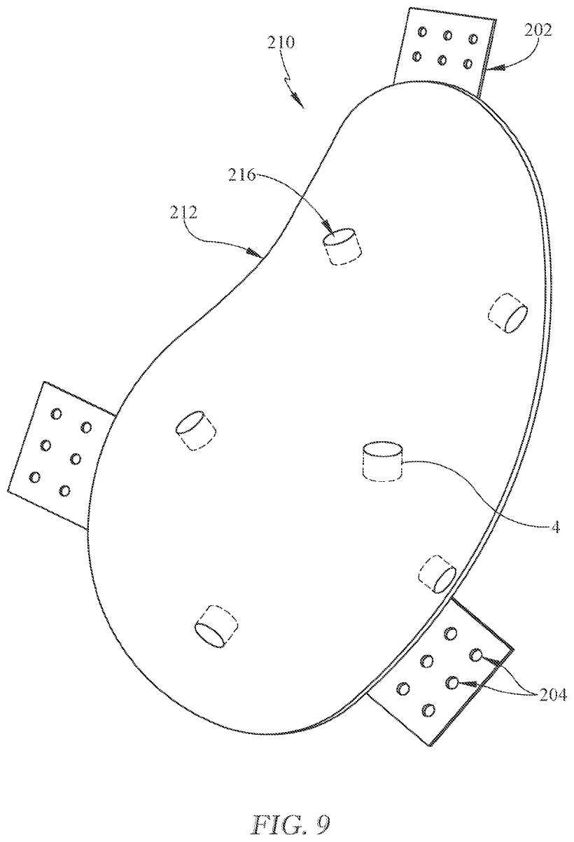

The positional-stabilizing well 16 of the implant 10 may be of a frustum shape defined by the side surface 40 and the bottom surface 42 as shown in FIG. 8. The frustum shape provides for ingrowth of the tissue 46 into the interior volume 44 sandwiched between the outermost surface 34 of the shell 12 and the innermost surface 43 of the shell 12 to secure against removal of the tissue 26 from the shell 12 by torsion or shear.

In some embodiments, the position-stabilizing wells 16 may have other suitable shapes. In some examples, ruffled or crenellated features may be incorporated. The wells 16 may be positively or negatively ribbed with features extending inwardly from side surfaces of the well. The wells 16 may be oval, triangular, asymmetrical, and/or irregularly shaped.

In an alternative illustrative embodiment, as shown in FIG. 9, an implant 210 can include a shell 212, with a plurality of position-stabilizing wells 216, and one or more position-stabilizing tabs 202. Each position-stabilizing tab 202 has one or more suture holes 204. Suturing the position-stabilizing tabs 202 can stabilized the implant 210 in a selected position upon implantation. Biodegradable suture can be used to suture the position-stabilizing tabs 202 through the holes 204 and attach the shell 212 to the underlying muscle 25.

The implant 10 may be a permanent or a temporary implant. For example, the implant 10 may be a tissue expander 10 that is are placed prior to a breast reconstruction surgery. The tissue expander 10 is slowly filled with saline, air, or other filler over several weeks until a desired skin volume is attained. After the desired skin volume is attained, the tissue expander 10 is removed and replaced by a permanent implant 10.

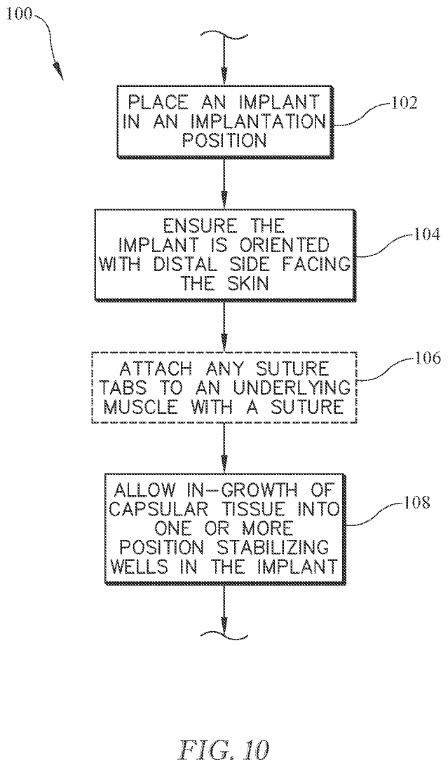

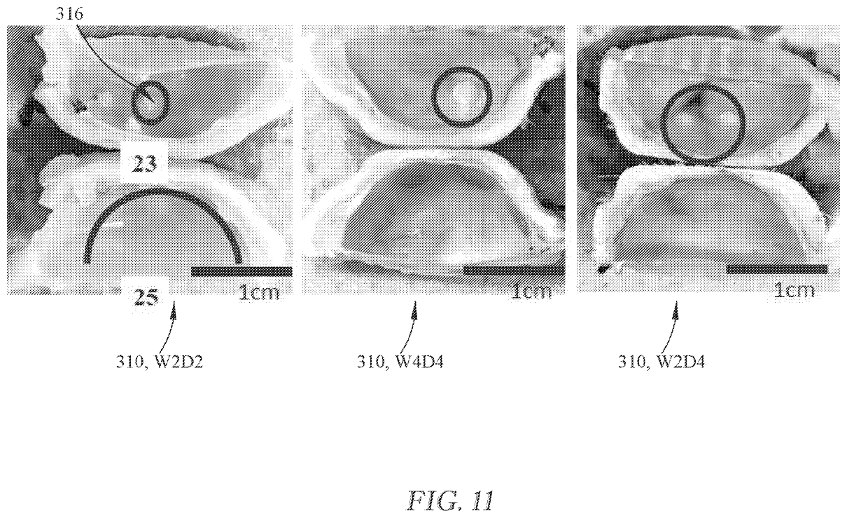

A method 100 of implanting the implant 10 or 210 is shown in FIG. 10. The method 100 begins at block 102. At block 102, the method 100 includes placing the implant 10 at the implantation position in the patient 11. Next, in block 104, the method 100 includes ensuring that the implant 10 or 210 is oriented with the distal panel 24 so that the distal panel 24 faces in kin 23 of the patient 11. Optionally, in block 106, the method 100 includes suturing the position-stabilizing tabs 202 to the underlying muscle 25 in the patient 11. Next, in block 108, the method 100 includes allowing the ingrowth of tissue 46 into the position-stabilizing wells 16 in the implant 10 or 210.

In another embodiment, an implant may not be limited to an anatomical shape. The implant may be round, spherical, crescenteric, or oval. Alternatively, the implant may be configured to be used at a different location in the patient (e.g., muscle implant, gluteal implant, calf implant). In some embodiments, the implant may be asymmetrical about any axis through the center of the implant.

### EXAMPLE 1

### Implant Fabrication

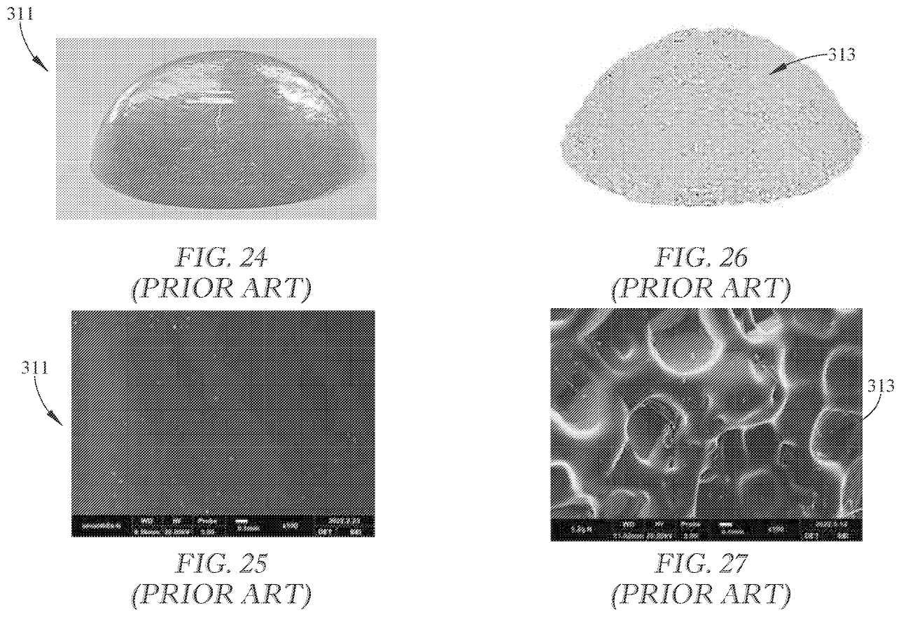

Three-dimensionally printed negative molds were produced using CAD modeling for implant casting. A 3D printed negative mold was printed for implant groups including a group comprising smooth implants 311 (see FIG. 24), three groups comprising textured implants 313 (see FIG. 26), and a group comprising implants 310 with a smooth surface and multiple position-stabilizing wells 316. Polydimethylsiloxane (PDMS) was used to fabricate hemispherical implants from the negative molds for each of the groups. Scanning electron microscopy was used to examine the surface characteristics of the implants 310, 311, 313 (see FIG. 4, FIG. 25, and FIG. 27).

### EXAMPLE 2

In vivo Study-Solid Round Implants

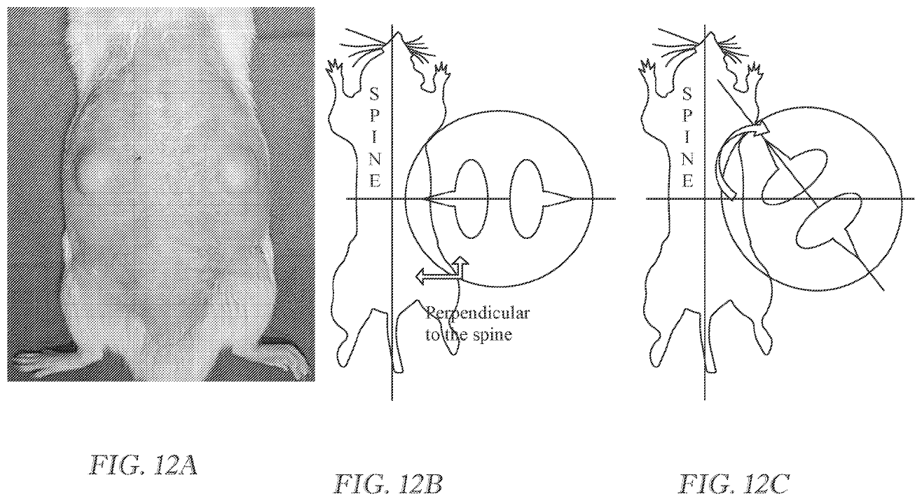

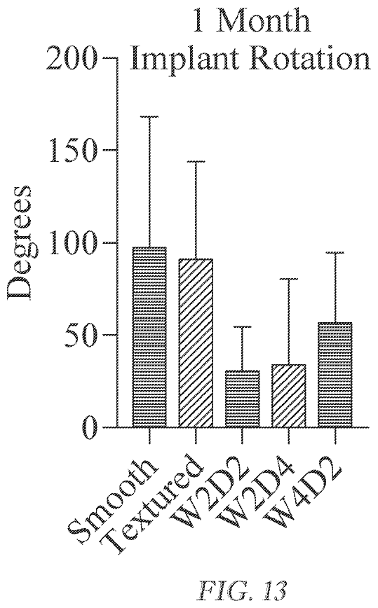

Hemispherical implants 310, 311, 313 were implanted using an in vivo rat model for one month and subsequently analyzed for degrees of implant rotation, total capsule 350 thickness, and gross histological analysis as shown in FIGS. 11-15. As shown in FIG. 11, the implants 310 with position-stabilizing wells 316 included implants 310 with position-stabilizing wells 316 that were 2 mm in width and 2 mm in height (W2D2), implants 310 with position-stabilizing wells 316 that were 4 mm in width and 2 mm in height (W4D2), and implants 310 with position-stabilizing wells 316 that were 2 mm in width and 4 mm in height (W2D4). All implants 310, 311, 313 were surgically implanted using sterile technique into a surgically created subcutaneous pocket on the dorsa of Sprague-Daley rats as shown in FIGS. 12A and 12B. Skin tattoos were used as a reference point to evaluate implant rotation. Degrees of rotation were measured relative to the rat spine at one month from time of placement as shown in FIG. 12C and FIG. 13. All implants 310 with position-stabilizing wells 316 rotated much less than either smooth implants 311 or textured implants 313. However, some implants flipped due to a relatively loose implantation pocket. Measurement of rotation had an inherent error due to movement of skin and animal growth.

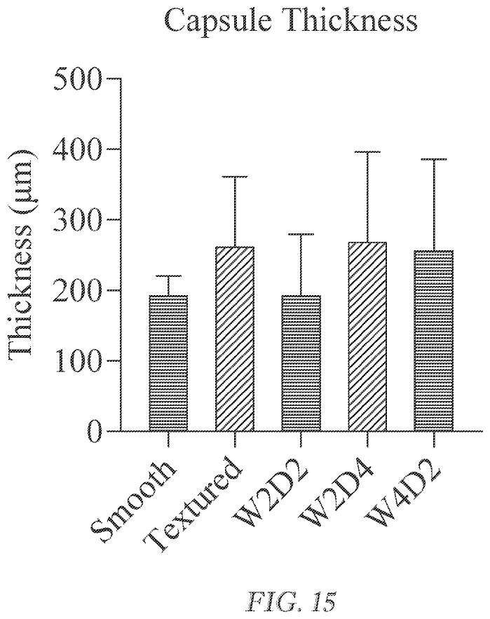

At the one month time point, the rats were sacrificed, and the implants 310, 311, 313 were en-bloc excised from the dorsum of the rats. Routine gross and H&E histologic staining of implant capsules 350 were obtained as shown in FIGS. 14 and 15. Ingrowth of tissue into the position-stabilizing wells 316 was noted in all implants 310.

### EXAMPLE 3

In vivo Study-Shaped Anatomic Implants

Teardrop shaped implants were fabricated to resemble the anatomically shape of the breast tissue. Solid PDMS implants were created and implanted in a rat model for one month and for three months. Degrees of implant rotation, total capsule thickness, and gross histological analysis were examined after 1 month and 3 months.

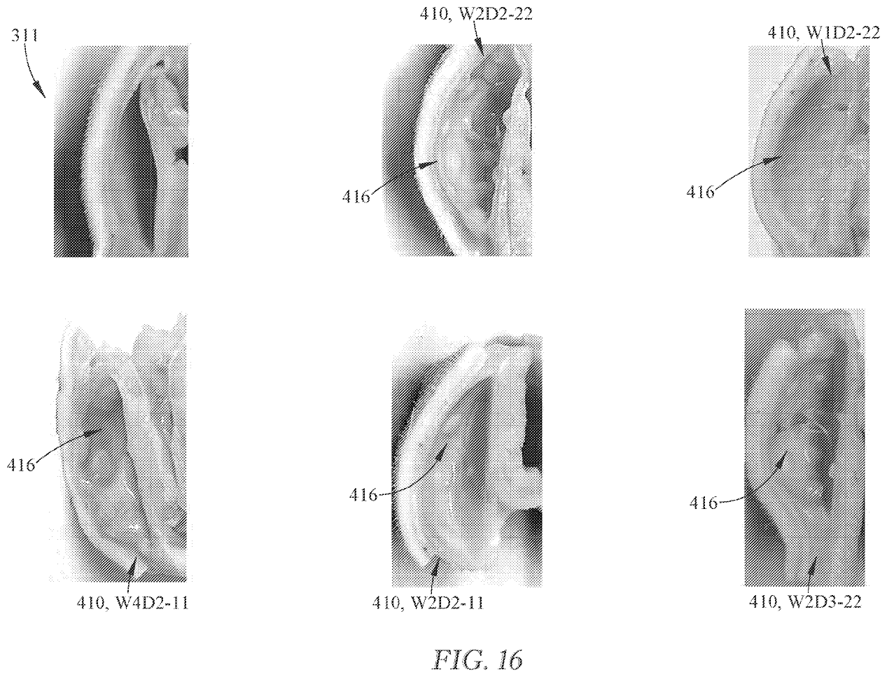

As shown in FIGS. 16-21, seven groups were studied including a group comprising smooth implants 311, a group comprising textured implants 313, and five groups comprising implants 410 with a smooth surface and multiple position-stabilizing wells 416. As shown in FIG. 16, the implants 410 with position-stabilizing wells 416 included implants 410 with 22 position-stabilizing wells 416 that were 2 mm in width and 2 mm in height (W2D2-22P), implants 410 with 22 position-stabilizing wells 416 that were 1 mm in width and 2 mm in height (W1D2-22P), implants 410 with 22 position-stabilizing wells 416 that were 2 mm in width and 3 mm in height (W2D3-22P), implants 410 with 11 position-stabilizing wells 416 that were 2 mm in width and 2 mm in height (W2D2-11P), and implants 410 with 11 position-stabilizing wells 416 that were 4 mm in width and 2 mm in height (W4D2-11P).

To overcome the inherent shortcoming of loose skin rat model (i.e. early implant movement with rat movement); three anchoring holes were included in each implant 410, 311, 313 which were used to secure the implant with a rapidly dissolving suture Polysorb (glycolide/lactide copolymer). It was hypothesized that initial fixation with a dissolvable suture would generate early stability during the first month post-implantation mirroring a tight implant surgical pocket, while ingrowth of tissue into the position-stabilizing wells 416 would generate long term implant stability after the first month.

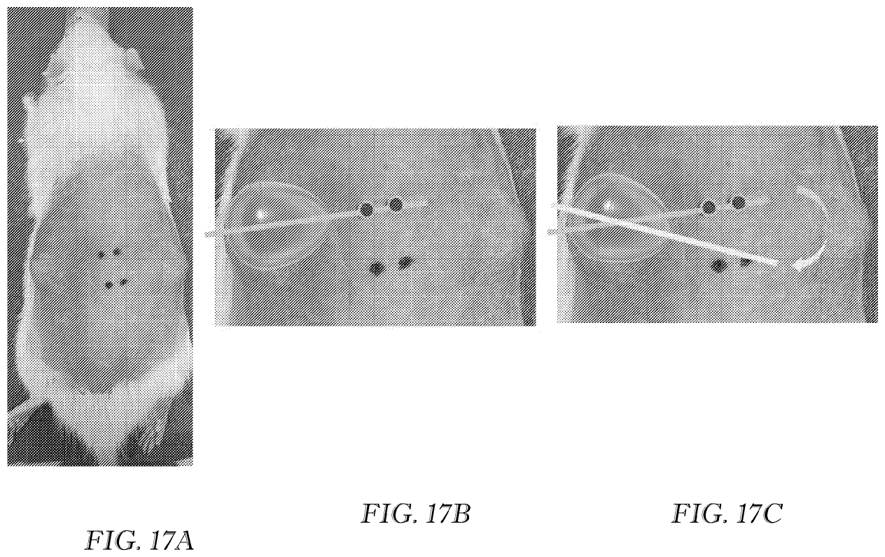

Two implants 410, 311, 313 were implanted on the dorsum of each rat as shown in FIGS. 17A and 17B. Using a permanent tattoo, the implant was aligned perpendicular to the apex of the anatomically shaped implant 410 or to implant 311, 313.

The degrees of rotation were measured from the initial position as marked by the tattoo at 1 month, 2 months, and 3 months post implantation (see FIG. 17C).

At the one month time point, the smooth implants 311 had a mean positional rotation of about 30.8±22° while the textured implants 313 had a mean rotation of about 7.5±2.8°. Implants 410 with 22 position-stabilizing wells 416 that were 2 mm in width and 2 mm in height had a mean rotation of about 15.8±5.3°, implants 410 with 22 position-stabilizing wells 416 that were 1 mm in width and 2 mm in height had a mean rotation of about 10±3.0, implants 410 with 22 position-stabilizing wells 416 that were 2 mm in width and 3 mm in height had a mean rotation of 10.8±4.2°, implants 410 with 11 position-stabilizing wells 416 that were 2 mm in width and 2 mm in height had a mean rotation of about 34.2±24.9° and implants 410 with 11 position-stabilizing wells 416 that were 4 mm in width and 2 mm in height had a mean rotation of about 12.5±3.2°.

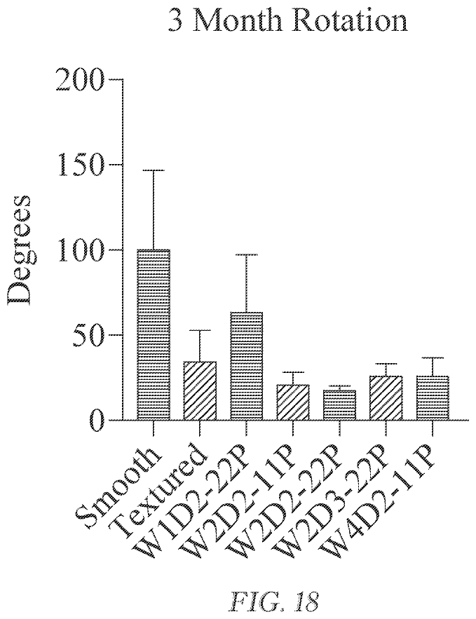

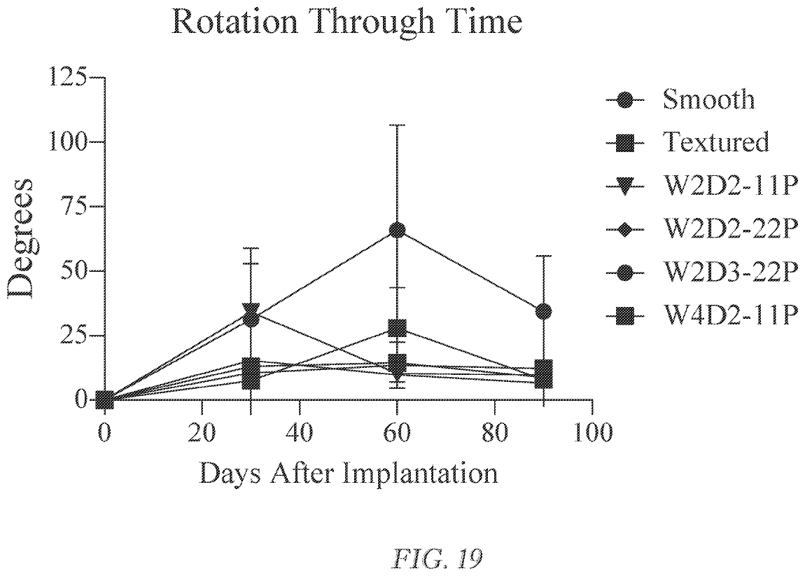

At the three month time point, the smooth implants 311 had a mean positional rotation of about 108.9±45.0° while the textured implants 313 had a mean rotation of about 43.1±15.4°. The mean positional rotation of the position-stabilizing wells 416 in the implants 410 at 3 months after implantation is shown in FIG. 18. Implants 410 with 22 position-stabilizing wells 416 that were 2 mm in width and 2 mm in height had a mean rotation of about 40.6±1.2°, implants 410 with 22 position-stabilizing wells 416 that were 1 mm in width and 2 mm in height had a mean rotation of about 75±35.0°, implants 410 with 22 position-stabilizing wells 416 that were 2 mm in width and 3 mm in height had a mean rotation of about 35±7.6°, implants 410 with 11 position-stabilizing wells 416 that were 2 mm in width and 2 mm in height had a mean rotation of about 70.6±40.7°, and implants 410 with 11 position-stabilizing wells 416 that were 4 mm in width and 2 mm in height had a mean rotation of about 37.5±13.2°. Furthermore, There was no statistically significant difference between the position of implants 410 with position-stabilizing wells 416 and textured implant 313 at the 3 month time point (p>0.05). Decreased stability was observed with lower position-stabilizing wells 416 width and with a lower number of position-stabilizing wells 416. The mean positional rotation of the position-stabilizing wells 416 in the implants 410 over a period of 3 months is shown in FIG. 19.

Temporary suture increased implant 410, 311, 313 stability particularly in implants 410 with position-stabilizing wells 416. Several temporary sutures pulled through the implants, which allowed early rotation. The skin tattoo measurement scheme had a built-in margin of error of about 20° to about 30°. Therefore any rotation of that amount or less may be considered no rotation

At the three month time point, the rats were sacrificed, and the implants 410, 311, 313 were en-bloc excised from the dorsum of the rats. Routine gross and H&E histologic staining of implant capsules was performed as shown in FIG. 20. Gross capsule 48 ingrowth into the position-stabilizing wells 416 was noted in all implants 410. Anchoring implants 410 to the underlying soft tissue created a stable surface allowing tissue ingrowth to be more consistent. Capsule thickness one month after the implants 410, 311, 313 is shown in FIG. 21. There were no significant differences in capsule 48 thickness between groups.

### EXAMPLE 4

In vivo Study-PDMS Shell/Silicone Implants

Implants 510 were designed to have a silicone shell with position-stabilizing wells 516 and which were then filled with a soft silicone gel 514 derived from commercially available breast implants resulting in an anatomically shaped miniature breast implant 510 with the highest fidelity to those used clinically in terms of construction and biomechanical qualities. An additional design change included suture tabs 502 that were impervious to suture pull through. Degree of implant rotation was examined after two weeks and after four weeks.

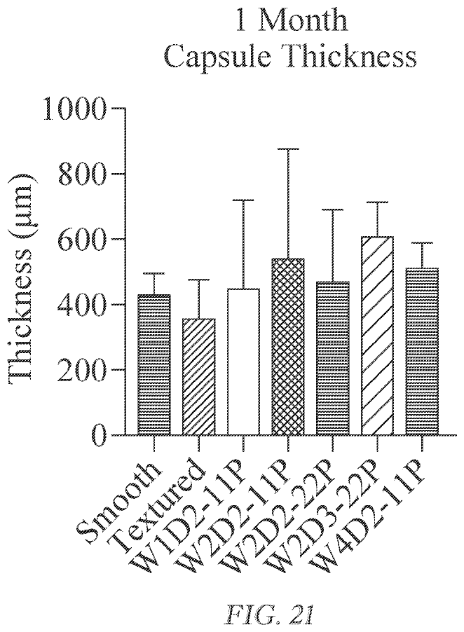

Seven groups were studied including a group comprising smooth implants 311, a group comprising textured implants 313, and five groups comprising implants 510 with a smooth surface and multiple position-stabilizing wells 516. As shown in FIG. 22, the implants 510 with position-stabilizing wells 516 included implants 510 with 22 position-stabilizing wells 516 that were 2 mm in width and 2 mm in height (W2D2-22P), implants 510 with 22 position-stabilizing wells 516 that were 1 mm in width and 2 mm in height (W1D2-11P), implants 510 with 22 position-stabilizing wells 516 that were 2 mm in width and 3 mm in height (W2D3-22P), implants 510 with 11 position-stabilizing wells 516 that were 4 mm in width and 2 mm in height (W4D2-11P), and implants 510 with 11 position-stabilizing wells 516 that were 2 mm in width and 2 mm in height (W2D2-11P).

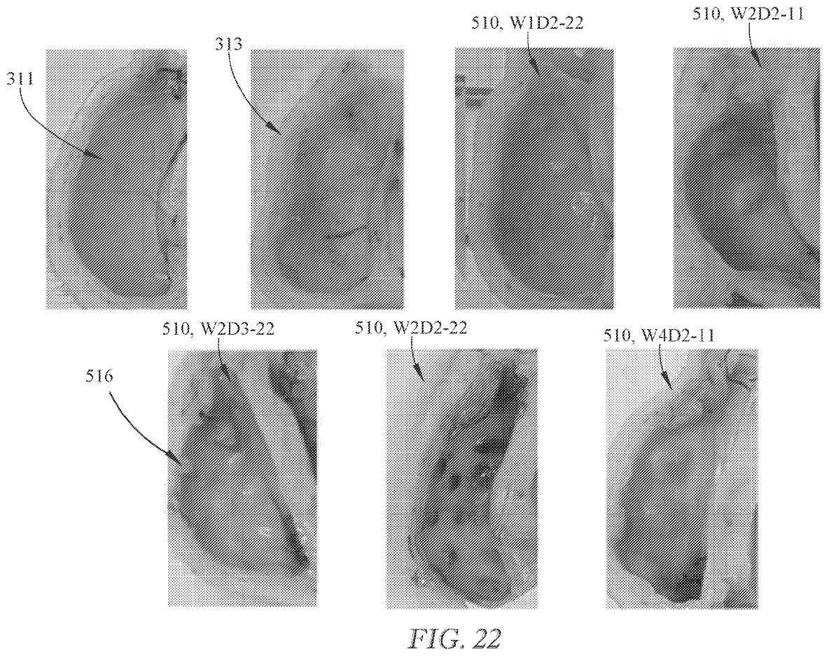

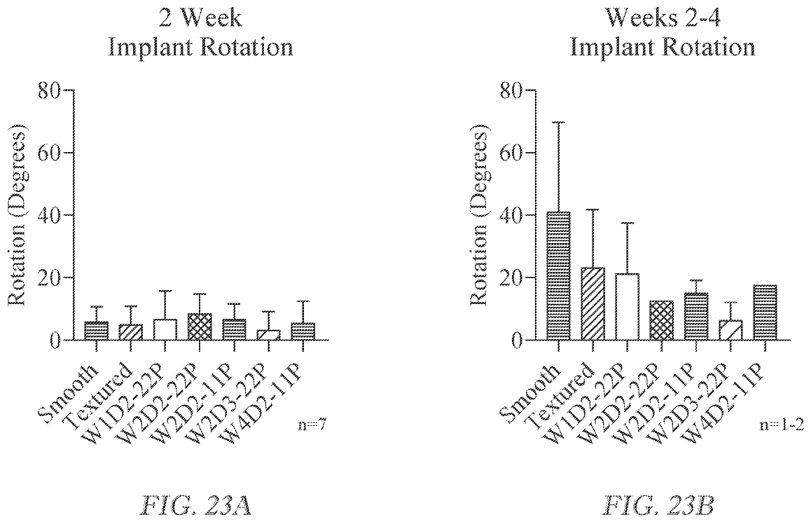

By the four week time point, there was a noticeable trend indicating increased stabilization in implants 510 with position-stabilizing wells 516 consistent with other data. The two week and four week post implantation rotation data is shown in FIGS. 23A and 23B suggesting improved stability in implants 510 with position-stabilizing wells 516 compared to smooth implants 510.

Those skilled in the art will understand that the devices and methods specifically described herein and illustrated in the accompanying drawings are non-limiting examples. The features illustrated or described in connection with one exemplary embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary.

The figures provided herein are not necessarily to scale, although a person skilled in the art will recognize instances where the figures are to scale and/or what a typical size is when the drawings are not to scale. Additionally, a number of terms may be used throughout the disclosure interchangeably but will be understood by a person skilled in the art. Further, to the extent features, sides, or steps are described as being “first” or “second,” such numerical ordering is generally arbitrary, and thus such numbering can be interchangeable. Lastly, the present disclosure includes some illustrations and descriptions that include prototypes or bench models. A person skilled in the art will recognize how to rely upon the present disclosure to integrate the techniques, systems, devices, and methods provided for into a product in view of the present disclosures.

1. A shaped breast implant, the implant comprising
a shell having a shell thickness, the shell shaped to be asymmetrical from bottom to top relative to a horizontal axis across a midsection of the shell with the shell having a bottom portion that is generally wider than a top portion to mimic the natural shape of breast tissue,
a filler that fills an interior space defined by the shell to give the shell a desired compliance, and
a plurality of position-stabilizing wells that extend from an outermost surface of the shell inwardly toward the gel, wherein each of the plurality of position-stabilizing wells are defined by an opening in the outermost surface of the shell at least one-half millimeter across and a depth configured to allow ingrowth of tissue into the shell that resists rotation of the implant relative to a patient so that the bottom of the shell remains below the top of the shell substantially in a selected position relative to the patient.

2. The implant of claim 1, wherein an exterior surface of the shell that defines the outermost surface of the shell as well as the position-stabilizing wells is smooth having a surface roughness of less than or equal to 4 μm so as to discourage development of a bacterial biofilm on the exterior surface.

3. The implant of claim 1, wherein the plurality of position-stabilizing wells are each defined by an opening at least one millimeters across.

4. The implant of claim 1, wherein the plurality of position-stabilizing wells are each defined by an opening less than or equal to four millimeters across.

5. The implant of claim 1, wherein the plurality of position-stabilizing wells are each defined by depth of less than or equal to four millimeters.

6. The implant of claim 1, wherein the plurality of position-stabilizing wells are of a cylindrical shape, wherein the cylindrical shape provides a uniform volume in each position-stabilizing well to allow ingrowth of the tissue to stabilize the implant.

7. The implant of claim 1, wherein the plurality of position-stabilizing wells are of a conical shape, wherein the conical shape provides a volume sufficient to allow ingrowth of the capsular tissue into the shell while managing reduction in the shell thickness closer to the gel.

8. The implant of claim 1, wherein the plurality of position-stabilizing wells are of a frustum shape, wherein the frustum shape provides for ingrowth of the tissue into a space sandwiched between the outermost and an innermost surface of the shell to secure against removal of the tissue from the shell.

9. The implant of claim 1, wherein the plurality of position-stabilizing wells are of a polygonal shape, wherein the polygonal shape provides more than one face that resists rotation of the tissue within the position-stabilizing well and manages removal of the tissue from the position-stabilizing well.

10. The implant of claim 1, wherein the outermost surface comprises a proximal outermost surface in contact with a muscle and a distal outermost surface in contact with skin when the implant is in the selected position, and wherein the implant comprises a different number of position-stabilizing wells on the proximal outermost surface than on the distal outermost surface.

11. The implant of claim 10, wherein the implant includes a larger number of position-stabilizing wells on the proximal outermost surface than on the distal outermost surface so as to provide increased attachment of the implant to the surrounding tissue and stabilize the implant relative to the patient.

12. The implant of claim 10, wherein the distal outermost surface has a larger surface area than the proximal surface and the implant includes a larger number of position-stabilizing wells on the distal outermost surface than on the proximal outermost surface so as to provide required attachment of the implant to the skin.

13. The implant of claim 1, wherein the bottom portion of the implant includes a larger number of position-stabilizing wells than the top portion so as to allow ingrowth of tissue into the position-stabilizing wells and stabilize the wider bottom portion of the implant.

14. A shaped breast implant, the implant comprising
a shell having a shell thickness, the shell shaped to be asymmetrical from bottom to top relative to a horizontal axis across a midsection of the shell with the shell having a bottom portion that is generally wider than a top portion to mimic the natural shape of breast tissue,
a filler that fills an interior space defined by the shell to give the shell a desired compliance, and
a plurality of position-stabilizing wells that extend from an outermost surface of the shell inwardly toward the gel, wherein an exterior surface of the shell that defines the outermost surface of the shell as well as the position-stabilizing wells is smooth, having a surface roughness of less than or equal to 4 μm so as to discourage development of a bacterial biofilm on the exterior surface.

15. The implant of claim 14, wherein each of the plurality of position-stabilizing wells are defined by an opening in the outermost surface of the shell at least one millimeter across and a depth configured to allow ingrowth of tissue into the shell that resists rotation of the implant relative to a patient so that the bottom of the shell remains below the top of the shell substantially in a selected position relative to the patient.

16. The implant of claim 14, wherein the plurality of position-stabilizing wells are each defined by an opening at least one millimeters across.

17. The implant of claim 14, wherein the plurality of position-stabilizing wells are each defined by an opening less than or equal to four millimeters across.

18. The implant of claim 14, wherein the plurality of position-stabilizing wells are each defined by depth of less than or equal to four millimeters.

19. The implant of claim 14, wherein the plurality of position-stabilizing wells are of
a cylindrical shape, wherein the cylindrical shape provides a uniform volume in each position-stabilizing well to allow ingrowth of the tissue to stabilize the implant;
a conical shape, wherein the conical shape provides a volume sufficient to allow ingrowth of tissue into the shell while managing reduction in the shell thickness closer to the gel;
a frustum shape, wherein the frustum shape provides for ingrowth of tissue into a space sandwiched between the outermost and an innermost surface of the shell to secure against removal of the tissue from the shell; and/or
a polygonal shape, wherein the polygonal shape provides a volume for ingrowth of tissue and more than one face of the polygonal shape resists rotation of the tissue within the position-stabilizing well and manages removal of the tissue from the position-stabilizing well.

20.-22. (canceled)

23. The implant of claim 14, wherein the outermost surface comprises a proximal outermost surface in contact with a muscle and a distal outermost surface in contact with skin when the implant is in a selected position in a patient, and wherein the implant comprises a different number of position-stabilizing wells on the proximal outermost surface than on the distal outermost surface.

24. The implant of claim 23, wherein the implant includes a larger number of position-stabilizing wells on the proximal outermost surface than on the distal outermost surface so as to provide increased attachment of the implant to the muscle and stabilize the implant relative to a patient.

25.-38. (canceled)

39. A method of implanting a breast implant in a patient comprising:
positioning the breast implant comprising a shell shaped to be asymmetrical from bottom to top relative to a horizontal axis across a midsection of the shell with the shell having a bottom portion that is generally wider than a top portion to mimic the natural shape of breast tissue in a selected position so that the bottom of the shell remains below the top of the shell substantially in the selected position relative to the patient; and
stabilizing the breast implant by allowing ingrowth of tissue into a plurality of position-stabilizing wells that extend from an outermost surface of the shell inwardly, wherein each of the plurality of position-stabilizing wells are defined by an opening in the outermost surface of the shell at least one millimeter across and a depth configured to allow the ingrowth of tissue into the shell that resists rotation of the implant relative to the patient.

40.-51. (canceled)
